(19) United States
(12) Patent Application Publication
(10) Pub. No.: US 2004/0147846 A1
(43) Pub. Date: Jul. 29, 2004

(54) External breast marker

(76) Inventors: Richard L. Mueller JR., Jackson, WY (US); Mallory Factor, New York, NY (US)

Correspondence Address: OLSON & HIERL, LTD., 36th Floor, 20 North Wacker Drive, Chicago, IL, 60606, US

(21) Appl. No.: 10/350,690

(22) Filed: Jan. 24, 2003

(51) Int. Cl. A61B 19/00; A61B 6/00

(52) U.S. Cl. 600/476; 600/478; 606/130; 128/897

(57) ABSTRACT

A device for mapping a mammary duct and suitable for recording and preserving patient biopsy information comprises an overlay that defines a nipple aperture sized to receive a breast nipple, an elongated window that extends radially outwardly from the nipple aperture, a lingulate region adjacent to the nipple aperture, and a mammary duct orifice map precursor situated in the lingulate region. At least a portion of the dorsal surface of the overlay is a markable surface.

### TECHNICAL FIELD OF THE INVENTION

The invention relates to a kit for mapping a mammary duct that terminates in a breast nipple orifice and method for using the kit.

### BACKGROUND OF THE INVENTION

Breast cancer is one of the health threats most feared by women, and is indeed the most common form of cancer in women. A key to treatment is early detection. For example, annual mammograms is a method that has been used in hopes of early detection of breast cancer. One problem with mammography is that such an imaging technique can only find breast cancer once it has taken form. All too often, breast cancer is discovered at a stage that is too far advanced, when therapeutic options and survival rates are severely limited. As such, more sensitive and reliable methods and devices are needed to detect cancerous, pre-cancerous, and other cancer indicators of the breast at an early stage. Such methods and devices could significantly improve breast cancer survival. While breast cancer is most common among women, in rare instances the human male may also have occurrences of breast cancer.

Other methods of detecting breast cancer are based on the fact that a vast majority of instances of breast cancer begins in the lining of mammary ducts. Studies have shown that fluid within the mammary duct contains high levels of breast cancer markers, and that an estimated 80%-90% of all breast cancers occur within the intraductal epithelium of the mammary glands. Fluid within the breast ducts contains an assemblage and concentration of hormones, growth factors and other potential markers comparable to those secreted by, or acting upon, the surrounding cells of the alveolar-ductal system. Likewise, mammary fluid typically contains cells and solid cellular debris or products that can also be used in cytological or immunological assays.

In a typical human breast, there are a plurality of mammary duct systems that terminate in individual mammary duct orifices at the nipple. Usually there are about 8 to 12 such duct orifices in each nipple. If a cytological or immunological assay reveals potential markers, further examination of the mammary duct or ducts is indicated. This further examination will likely comprise taking a biopsy of mammary duct tissue. One method of obtaining a sample of mammary duct tissue is through use of a microendoscope that may include a sampling component, an illumination component, and a viewing component. After a sample of mammary duct tissue is obtained, an analysis of the sample is performed.

Typically, a number of days or even weeks will pass between the mammary duct tissue biopsy and the completion of the assay. As such, creating a record of the location of the mammary duct examined, and the biopsy location for further examination, if necessary, is desired. It is also desired to maintain a record of the patient's information, including the patient's name, the date of the biopsy and the mammary duct sampled. It may further be advantageous to utilize photographs taken via an endoscope with the record so as to provide additional information regarding the examination. Patient files are typically flat files. Therefore, it is desired that the device for mapping the mammary duct examined and the biopsy location lie flat to fit in a typical patient file.

### SUMMARY OF THE INVENTION

A device for mapping a mammary duct that terminates in a breast nipple orifice is provided. The device comprises an elongated overlay of a pliant material which defines a nipple aperture that is sized to receive a human breast nipple. Preferably the pliant material is transparent. The overlay has a dorsal surface, which faces away from the patient, and a ventral surface, which contacts the patient's breast. The overlay further includes an elongated window extending radially outwardly from the nipple aperture. The window may either be a transparent region of the overlay, or may be a slot in the overlay.

Preferably, the overlay includes a pressure sensitive adhesive on the ventral surface for temporary securement of the overlay to the patient's breast. At least the ventral surface of the overlay further preferably includes an antimicrobial agent. Suitable antimicrobial agents are antibiotics, especially antifungal compounds such as the polyenes, e.g., Nystatin, Natamycin, and the like; the allylamines, e.g., Naftifine, Terbinafine, and the like; the immidazoles, e.g., Ketoconaizole, Miconazole, and the like; the triazoles, e.g., Fluconazole, Itraconazole, and the like; the morpholines, e.g., amorolfine, and the like. The antimicrobial agents can be applied to the surfaces of the overlay dissolved in a volatile solvent such as ethanol and the like, or can be incorporated into the pliant overlay material itself.

The overlay is further configured with a lingulate region i.e., adjacent to the nipple aperture. A mammary duct orifice map precursor is provided on the lingulate region so that a physician may map the nipple orifice and corresponding mammary duct examined. Preferably, the lingulate region is unitary with the overlay. Alternatively, the lingulate region may be attached to the overlay by an adhesive.

The dorsal surface of the overlay includes at least one writable region. In particular, the mammary duct orifice map precursor has a markable surface suitable for writing thereon. It is preferred that the entire dorsal surface of the overlay be markable.

Preferably, the overlay further includes a slit that extends inwardly from a periphery of the overlay to the nipple aperture. The overlay may thereby be placed about a nipple during other procedures, such as a visual inspection of the patient's mammary duct with a microendoscope, by passing the microendoscope through the slit such that the overlay circumscribes the nipple.

It is also preferred that distance indicia are provided on the overlay along the elongated window thereof, and that azimuth indicia are provided on the overlay about the nipple aperture.

In order to map and record the biopsy site in a mammary duct, and to record the patient's biopsy information, the location of the biopsy site is illuminated. This can be done through use of a microendoscope having an illuminator, such as a fiber optic assembly. When the illuminator is energized, the light emitted therefrom is sufficient to shine through the skin and to be visible on the surface of the breast as an illuminated area. The overlay is positioned on the breast so that the nipple aperture circumscribes the nipple either before the biopsy, or, as discussed, the microendoscope is slipped past the slit if the overlay is placed while the microendoscope or other instrument is being used within the mammary duct. The overlay is oriented relative to the patient such that the illuminated area is visible through the window. A marking is made on the overlay adjacent to the window to indicate the illuminated area such as with an indelible marker, or with a sticker. The physician also marks the overlay adjacent to the nipple aperture to indicate alignment of the overlay relative to the patient's body. For example, the physician may write the word “HEAD” on the overlay to indicate the direction of the patient's head relative to the overlay. This provides a frame of reference for repositioning of the overlay for a subsequent examination, as discussed below.

As an endoscope is passed through a mammary duct, it is preferred that photographs of the intra-ductal region be taken at regular intervals. The intervals at which photographs are taken can correspond with distance indicia on the overlay and be suitably so marked. Such photographs can be included in a patient portfolio along with the overlay as part of the patient's record. Photographs may also be taken with the endoscope positioned outside the mammary duct, yet with an introducer still disposed within the mammary duct to record the angle of the instrument relative to the breast when relaxed.

The mammary duct orifice map precursor can be marked to indicate the region of the nipple, including the mammary duct examined. The patient's name, and other pertinent information, such as the date of the biopsy, weight of the patient, the stage of the patient's menstrual cycle, etc., may also be entered on the overlay. The overlay may further be marked by the physician with an approximate track of the path traveled by the endoscope. The overlay may then be removed from the patient, and saved in the patient's file.

In an alternate embodiment, the mapping device is provided as a kit suitable for mapping a mammary duct and suitable for recording and preserving patient biopsy information. The kit is constituted by several components. When the components are assembled, a layered mammary duct mapping overlay is formed with the components thereof integral with one another and similar to the unitary overlay discussed above.

One kit component is an external target marker for marking on an external surface of a human breast a location that corresponds to a site in the mammary duct. The external target marker may be any means of marking the external surface of a human breast, such as an adhesive sticker, or a temporary dye or ink. Preferably, the external target is a thin circular plastic piece with an adhesive backing for removably securing the external target to the human breast. The kit of the present invention can include one or more such external target markers. The target can also have varying degrees of translucence, or opacity, to record by matching additional information such as the approximate intensity of light transmitted through the skin inasmuch as the depth of the region of interest can be estimated from the transmitted light intensity.

Another component of the kit is a nipple alignment marker. The nipple alignment marker is positioned proximate to a human breast nipple, and includes at least one body part alignment indicator or a markable region where such alignment can be indicated. The body part alignment indicator preferably indicates the direction of the patient's head or other body part, e.g., feet, relative to the nipple such that the alignment indicator can be repeatedly properly oriented to reliably reproduce original alignment. In a specific example, the nipple alignment marker is arranged such that the head and feet indicators on the nipple alignment marker have the same orientation as the patient's head and feet.

The nipple alignment marker can have any variety of shapes; however, it is preferable that the nipple alignment marker circumscribes the nipple. Given the shape of the typical human breast, the nipple alignment marker is preferably circular or disc-shaped with a through opening for the nipple. The nipple alignment marker also preferably includes an adhesive backing both sides thereof. The adhesive on one side allows the nipple alignment marker to be temporarily and securely positioned on the patient, while the adhesive backing on the side opposite the one side, as explained below, permits the layered mammary duct mapping assembly to be more easily assembled. The nipple alignment marker preferably includes a slit that extends inwardly from a periphery of the nipple alignment marker to the nipple aperture. In this manner, the nipple alignment marker can be placed about a nipple during medical procedures, such as a visual inspection of the patient's mammary duct with a microendoscope. The slit permits placement of the overlay to circumscribe the nipple while the microendoscope is already in place in the nipple. While the nipple alignment marker may be made of any suitable material, it is preferably made of a physiologically compatible plastic material such as polyethylene, polypropylene, and the like.

A pliant alignment slide overlay is also part of the kit embodying the present invention. The alignment slide overlay is conformable to a human breast contour and when in position lies along the curvature of a human breast. Preferably the alignment slide overlay is sufficiently pliant and light in weight so that it does not deform the breast to a material degree during the procedure. The alignment slide overlay is preferably made of a polyurethane, a plasticized elastomeric material, and the like, and is substantially transparent. The alignment slide overlay may be secured to the nipple alignment marker by way of an adhesive on the distal side of the nipple alignment marker relative to the breast. The adhesive can be protected prior to use by a release strip and exposed for use by removing a release strip. The alignment slide overlay further defines a nipple aperture and an elongated window such as a transparent region of the alignment slide overlay or a slot in the alignment slide overlay. The alignment slide overlay also preferably has a writable surface on its dorsal (or proximal) side.

A mammary duct orifice map precursor is securable to the alignment slide overlay, preferably by way of an adhesive. The orifice map precursor may be a rectangular grid, or the like to enable the physician or other medical personnel to mark the region of the nipple that includes the orifice of the mammary duct examined. The map precursor preferably also includes space for entering the patient's name and date of examination. The map precursor can also include at least one body part alignment indicator or a markable region for such indication. When a transparent map precursor is permanently mounted over the nipple alignment marker, the body part alignment indicator of the nipple alignment marker is visible relative to the body part alignment indicator on the mammary duct orifice map precursor and no additional alignment indicator is necessary. When a body part alignment indicator is present on the map precursor as well as the nipple alignment marker, the physician or medical personnel aligns the body part alignment indicators on both of the nipple alignment marker and the map precursor as the mammary duct mapping assembly is assembled during examination or biopsy. For example, the map precursor may include a cut out window or window that, when the map precursor is properly oriented and placed over the alignment slide overlay and nipple alignment marker, allow the body part alignment indicators to be seen at the same time. Alternatively, the map precursor can be made such that the portion overlapping the nipple alignment marker is transparent, as stated hereinabove.

The distal side of the nipple alignment marker and the bottom side of the map are bonded to the alignment slide by adhesive to form a layered mammary duct mapping overlay. In other words, the alignment slide is secured between the nipple alignment marker and the outer cover to form a permanent layered overlay. Alternatively, if no adhesive backings are used, the components may be clipped, stapled or otherwise held together.

The assembly and use of this alternate embodiment of the present invention is similar to that discussed with the integrally formed overlay. As discussed before, the location of the biopsy site is first illuminated. The external target marker is placed over the illuminated area indicating the location of the biopsy. After the biopsy procedure is completed, the nipple alignment marker is then placed proximate to the nipple. For accuracy, it is preferred that the nipple alignment marker has a configuration that circumscribes the nipple. Similar to the external target marker, the nipple alignment marker is preferably removably secured to the breast with an adhesive. Before securing the nipple alignment marker to the breast, however, a body part alignment indicator on the nipple alignment marker is aligned with the corresponding body part. For example, the body part alignment indicator may be the word “HEAD,” which should be aligned with the patient's head. Preferably, there may be a plurality of body part alignment indicators, such as “HEAD” and “FEET,” which will be aligned, in this example, with the patient's head and feet. This will provide a frame of reference for the nipple alignment marker for a later examination as discussed below.

The alignment slide overlay, having an elongated window, is then positioned over the nipple alignment marker and the external target marker. The location of the target can also be indicated by any appropriate marking on the alignment slide overlay or may simply be aligned with an end of the elongated window. The alignment slide overlay is also preferably secured to the nipple alignment marker, such as by an adhesive on the proximal side of the nipple alignment marker relative to the breast. In so doing, the distance from the target to the nipple alignment marker can be memorialized.

If desired, a series of photographs may be taken by the physician with the endoscope as the endoscope is passed through the mammary duct. The photographs preferably are taken so that as the illuminated area on the breast surface can be correlated with distance indicia on the alignment slide overlay. Such photographs can be stored in the patient's portfolio as well.

The map precursor is permanently secured to the alignment slide overlay. As such, the nipple alignment marker, the alignment slide overlay, and the map precursor are bonded together and form a layered mammary duct mapping assembly wherein the alignment slide is sandwiched between the nipple alignment marker and the map. As such, the length and direction of the alignment slide extending from the nipple alignment marker towards the external target is fixed. Also, the alignment of the parts relative to the patient's head is known because of the body alignment indicators on the nipple alignment marker.

The map precursor is marked to indicate the region of the nipple including the mammary duct examined as before. The entire layered mammary duct mapping overlay may then be removed from the patient, and saved in the patient's file. The external target marker can also be removed at this time, if desired. Alternatively, the external target marker can remain in place until the patient's return visit. Similar to the unitary embodiment of the present invention described hereinabove, it is preferred that the components of this alternate embodiment also includes an anti-microbial agent

When the patient returns for further examination, the region from which the tissue sample was taken can be readily determined by replacing the overlay over the appropriate nipple, and aligned with, for example, the patient's head. The target section of the alignment slide will then correspond with the surface area of the breast which was illuminated to indicate the biopsy region.

A microendoscope having an illumination feature and a viewing feature can then be inserted for further examination into the mammary duct previously examined through the duct orifice as indicated on the mammary duct map. The illuminated end of the microendoscope is visible through the skin of the patient and can thus be positioned in the region indicated by the external target marker. The physician will thereby have a good indication of the location of the earlier biopsy and can appropriately focus therapeutic treatment, if indicated.

### BRIEF DESCRIPTION OF THE DRAWINGS

In the drawings,

FIG. 1 is a schematic illustrating examination of a breast mammary duct with a microendoscope;

FIG. 2 illustrates a preferred embodiment of a mapping device of the present invention;

FIG. 2A illustrates the mapping device of FIG. 2 applied to a human breast;

FIG. 3 illustrates a kit embodiment of the mapping device embodying present invention constituted by a mammary duct orifice map precursor, an alignment slide, a nipple alignment marker, and an external target marker;

FIG. 4 is a perspective view of a preferred embodiment of an external target marker;

FIG. 5 is a perspective view of a preferred embodiment of a nipple alignment marker;

FIG. 6 is a perspective view of a preferred embodiment of the nipple alignment marker and external target marker applied to a human breast;

FIG. 7 is a perspective view of a preferred embodiment of a alignment slide overlay;

FIG. 8 is a perspective view of a preferred embodiment of the alignment slide overlay, nipple alignment marker, and external target marker assembled on a human breast;

FIG. 9 is a perspective view of a preferred embodiment of a mammary duct orifice map precursor;

FIG. 10 is a perspective view of a preferred embodiment of the mammary duct orifice map precursor, alignment slide overlay, nipple alignment marker, and external target marker assembled on a human breast; and

FIG. 11 is a schematic perspective view of a patient portfolio.

### DESCRIPTION OF THE PREFERRED EMBODIMENT OF THE INVENTION

The invention disclosed herein is, of course, susceptible of being embodied in many different manners. Shown in the drawings and described herein below in detail is a preferred embodiment of the invention. It is to be understood, however, that the present disclosure is an exemplification of the principles of the invention and does not limit the invention to the illustrated embodiments.

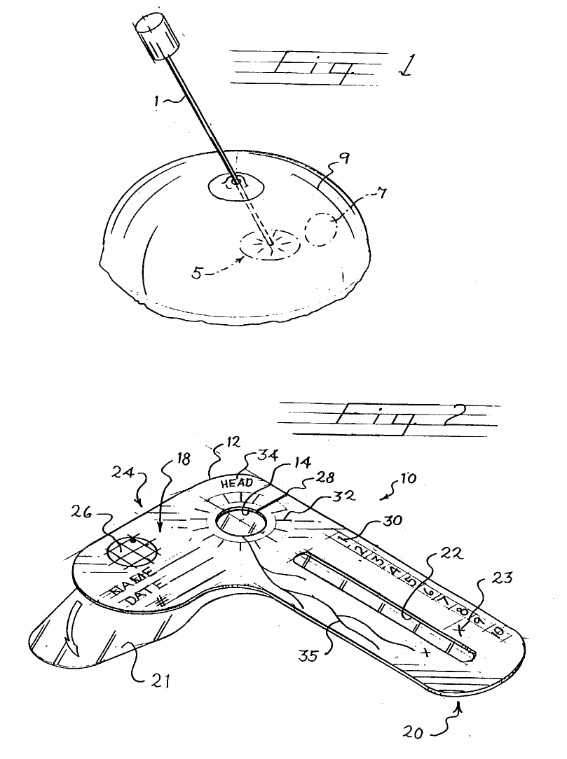

FIG. 1 illustrates a microendoscope 1 used to examine a mammary duct. Microendoscope includes an illuminator 3. When the illuminator 3 is energized, the emitted light illuminates internal tissue region 5. Light also shines through the patient's skin, and is visible also as an illuminated area 7 on the surface of the breast 9. To mark the location of the illuminated area 7, and thereby provide an indication of the location of the illuminated internal tissue region 5, the present invention is applied to the patient.

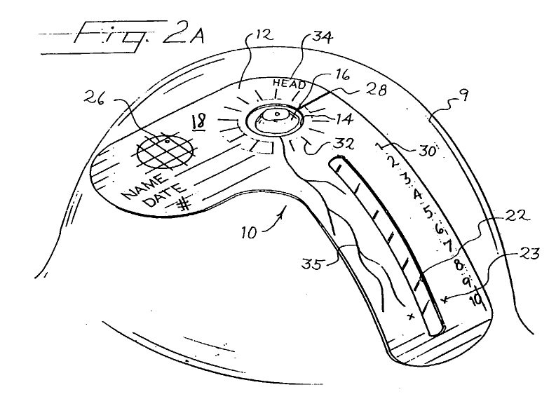

FIGS. 2 and 2A illustrate a preferred embodiment of the present invention. The device 10 comprises an elongated overlay 12 of a pliant material, which defines a nipple aperture 14 sized to receive a human breast nipple 16 (FIG. 2A). Preferably the pliant material is transparent. The overlay 12 has a dorsal surface 18 bearing various markings 30, 32, and 34, and which faces away from the patient. A ventral surface 20 of overlay 12, opposite to dorsal surface 18, is adapted to contact the patient's breast 9 (FIG. 2A). The overlay 12 further includes an elongated window 22 extending radially outwardly from the nipple aperture 14. The window 22 extends radially outwardly from nipple aperture 14, and can be a transparent region of the overlay 12 or a slot in the overlay 12. The illuminated area 7 (FIG. 1) is viewable through window 22 and the physician places on the overlay 12 the general location of the illuminated area and marks the illuminated area with an indicator 23, such as by marking the dorsal surface 18 with an “x” as shown or by placing a sticker thereon. It is preferred that the entire dorsal surface 18 be markable.

Preferably, the overlay 12 includes a pressure sensitive adhesive on the ventral surface 20 and protected by a release strip 21. The pressure sensitive adhesive is exposed by removing a release strip 21. The ventral surface 20 of the overlay 12 further preferably includes an antimicrobial agent.

The overlay 12 is further configured with a lingulate region 24, such as a tab or a tongue, adjacent to and extending from the nipple aperture 14. On the lingulate region 24 is a mammary duct orifice map precursor 26 on which a physician can note the nipple orifice (e.g., with a dot 27) and corresponding mammary duct examined (e.g., with a tracing 35). Preferably, the lingulate region 24 is unitary with the overlay 12. Alternatively, the lingulate region may be attached to the overlay by an adhesive or the like expedient.

The overlay 12 further preferably includes a slit 28 which extends inwardly from periphery of the overlay 12 to the nipple aperture 14. The overlay 12 thus can be placed about a nipple 16 during a visual inspection of the patient's mammary duct with a microendoscope 1 (FIG. 1), and while the microendoscope is in place, by passing the microendoscope 1 through the slit 28.

Distance indicia 30 can be provided on the overlay along the elongated window 22 thereof, and azimuth indicia 32 are provided on the overlay 12 about the nipple aperture 14.

The overlay 12 is oriented relative to the patient such that the illuminated area is visible through the window 22. A marking 23 is made on the overlay 12 to indicate the illuminated area such as with an indelible marker, or with a sticker. The physician may also draw a sketch 35 of the examined mammary duct path on the overlay if desired. The physician also provides a body alignment part indicator 34 to mark the alignment of the overlay 12 relative to the patient's body. For example, the physician may write the word “HEAD” on the overlay 12 to indicate the direction of the patient's head relative to the overlay when in place. This provides a frame of reference for the overlay 12 for a later examination as discussed below. After initial examination, the overlay 12 is removed from the patient and placed in the patient's file for further reference.

FIG. 3 illustrates an alternate embodiment of the present invention in the form of a kit 100 constituted by an external target marker 110, a nipple alignment marker 120, an alignment slide overlay 130, and a mammary duct orifice map precursor 140. The kit 100 may also include accessory items such as marking pens or sterile wipes (not shown). Preferably, kit 100 comprises a base 102 provided with appropriate sockets that receive the external target marker 110, nipple alignment marker 120, alignment slide overlay 130, and the mammary duct orifice map precursor 140, as well as any optional items. A transparent removable cover 103 for base 102 can also be provided if desired.

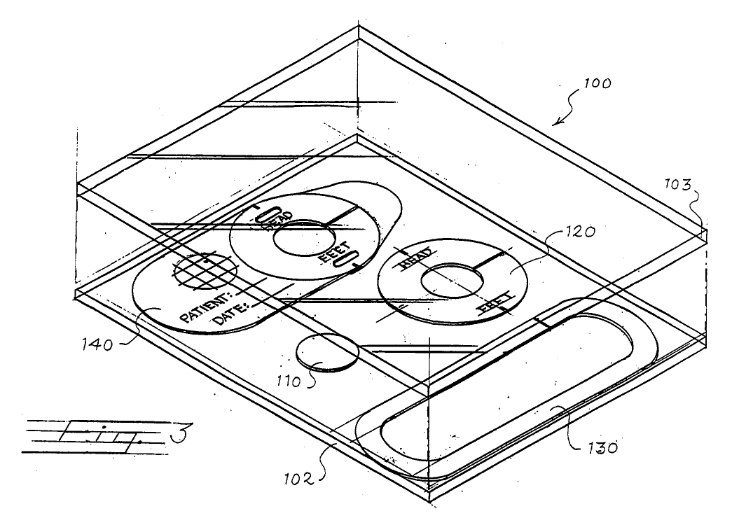

FIG. 4 illustrates a preferred external target marker 110 having dorsal or proximal side 111 and a ventral or distal side 112. External target marker 110 also preferably includes an adhesive backing layer on distal side 112 and a release strip 113 for the adhesive backing layer. Release strip 113 is removed prior to use. Preferably, the external target marker 110 is made of a transparent or translucent material. The external target marker 110 may also include markings 114 thereon if desired.

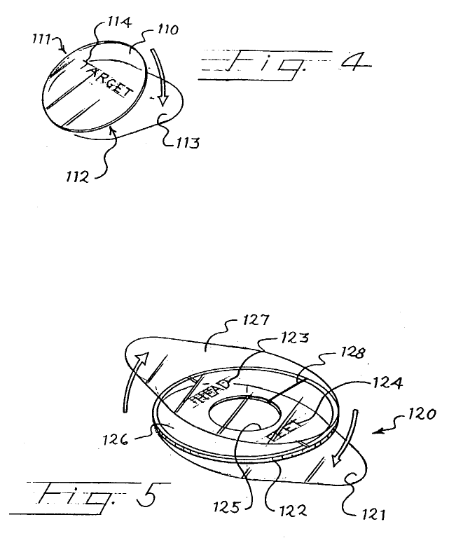

As shown in FIGS. 5 and 6, nipple alignment marker 120 is preferably placed so as to circumscribe the nipple 160 (FIG. 6). Prior to securing the nipple alignment marker 120 on the breast 161′ the body part alignment indicators 123 and 124 are oriented relative to the indicated body part or parts of the patient. For example, body part alignment indicator 123 is aligned with the patient's head (not shown), and body part alignment indicator 124 is aligned with the patient's feet (not shown). Nipple 160 extends through opening 125 and the adhesive backing on the distal side 126 removably secures the nipple alignment marker 120 to the breast 161. Opening 125 is preferably sized such that the nipples of a wide range of patients may be accommodated. Nipple alignment marker 120 further preferably includes a slit 128 extending from the periphery of nipple alignment marker 120 to the opening 125.

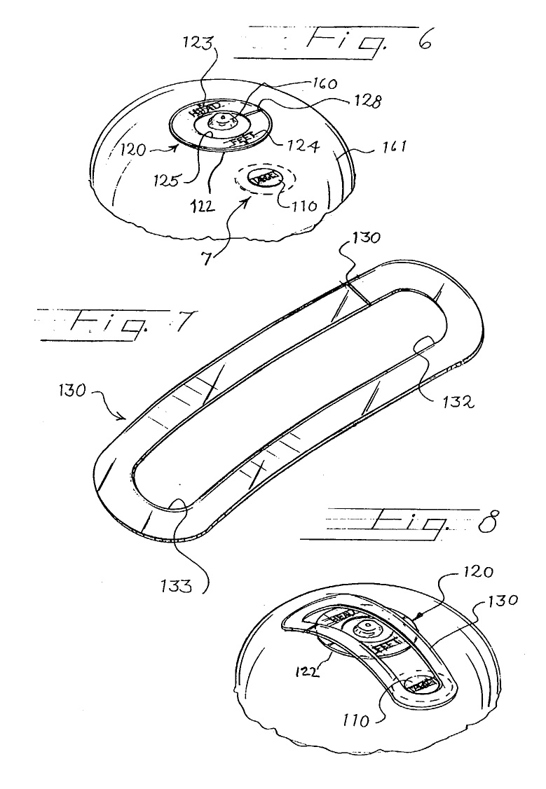

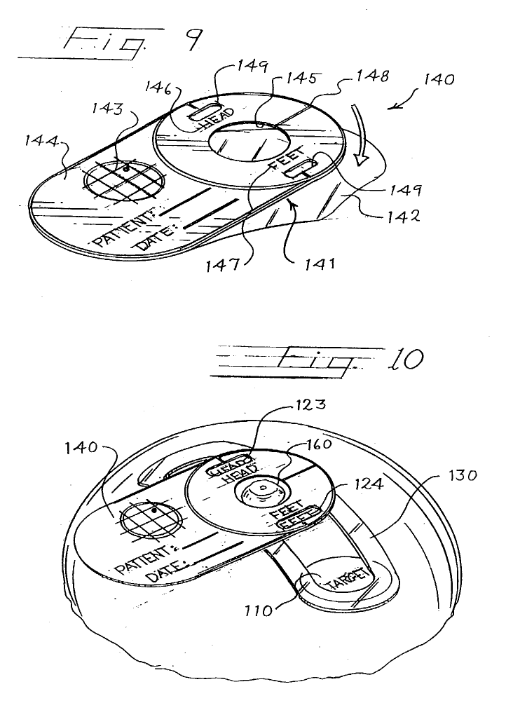

As shown in FIGS. 7 and 8, the alignment slide overlay 130 having an integrally formed elongated window 133 is then placed over the nipple alignment marker 120 and the external target marker 110. Elongated window is sufficiently large to accommodate the diameter of nipple 160 at end 132. Alternatively, the alignment slide overlay 130 can include a nipple aperture and a window substantially as shown in FIGS. 2 and 3.

In so doing, the distance from the external target marker 110 to the nipple alignment marker 120 and thus nipple 160 can be memorialized. Alternatively, distance indicia can be provided on overlay 130 as discussed hereinabove with respect to the unitary embodiment shown in FIGS. 2 and 3. The alignment slide overlay 130 is also preferably secured to the nipple alignment marker 120, such as by an adhesive on the distal side 126 of the nipple alignment marker 120. The alignment slide overlay 130 further preferably includes a slit 135 which extends from the periphery of the alignment slide overlay 130 to the nipple aperture 132.

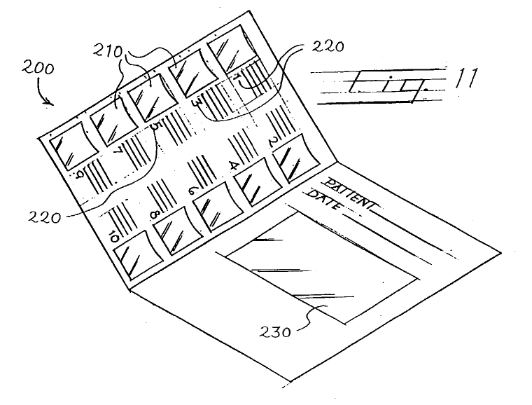

A mammary duct orifice map precursor 140 is provided as shown in FIGS. 9 and 10. Map precursor 140 is securable to the alignment slide overlay 130, preferably by way of an adhesive on the distal side 141 of the map precursor 140, which is exposed prior to use by removing release strip 142. Grid 143 is provided on the distal side 144 of the map precursor 140. Grid 143 may be a rectangular grid with sections to enable the physician or other medical personnel to indicate with a mark 150 on the map precursor 140 the location of the nipple orifice for the mammary duct examined. The dorsal or proximal side 144 of the map precursor 140 has a surface suitable for writing. The map precursor 140 can also include space for the patient's name and date of examination to be indicated. Opening 145 is sized to receive the nipple and substantially corresponds to the opening 125 in the nipple alignment marker 120 through which the nipple 160 extends. A slit 148 is provided from the periphery of map precursor 140 to the opening 145. Also, at least one body part alignment indicator such as 146 and 147 is preferably provided on map precursor 140. When the map precursor 140 is placed over the nipple alignment marker 120 the body part alignment indicators 123 and 124 of the nipple alignment marker 120 are visible relative to body part alignment indicators 146 and 147 on the map precursor 140. As shown, the map precursor 140 includes cut out windows 148, 149 so when the map precursor 140 is properly oriented and placed over the alignment slide overlay 130 and nipple alignment marker 120 the body part alignment indicators 123 and 124 on the nipple alignment marker 120 are visible.

Before or during the medical procedure the grid 143 on the map precursor 140 is marked to indicate the approximate location of the nipple orifice for the mammary duct examined. The patient's name, weight, date in menstrual cycle, and other pertinent information, such as the date of the biopsy, may also be entered on the margin of map precursor 140.

The thus produced parts assembly, i.e., nipple alignment marker 120, alignment slide overlay 130, and map precursor 140, are bonded together to form a layered mammary duct mapping overlay wherein the alignment slide overlay 130 is sandwiched between the nipple alignment marker 120 and the map precursor 140. The entire layered assembly may then removed from the patient and saved in the patient's file for future reference.

A photographic record of the examination may also be created using either the assembled kit, or the unitary overlay, and saved in a patient portfolio 200 as shown in FIG. 11. The patient portfolio 200 preferably includes holders or pockets 210 for photographs taken via the endoscope during the examination of the mammary duct. Preferably, the holders 210 have markings 220 that correspond to distance indicia on the overlay utilized. It is preferred that portfolio 200 includes a pouch 230 for the overlay as well. The patient portfolio 200 can be placed in the patient's file as part of the patient's medical record.

During a return visit, the region from which the tissue sample was taken or which was examined can be re-examined, if necessary, using the overlay as a guide. In order to determine the proper location, the mapping overlay, either the unitary overlay or the assembly, is placed over the nipple, and aligned with the patient's head and feet. The markings on the overlay will then correspond with the surface area of the breast which was illuminated to indicate the biopsy region or examined region. A microendoscope having an illumination feature and a viewing feature can then be inserted into the mammary duct previously examined through the duct orifice indicated on the mammary duct map. The illuminated end of the microendoscope will be visible through the skin of the patient and can thus be positioned in the desired region. The physician will thereby have a good indication of the location of the region of interest.

The foregoing description is to be taken as illustrative, but not limiting. Still other variants within the spirit and scope of the present invention will readily present themselves to those skilled in the art.

We claim:

1. A device for mapping a mammary duct that terminates in a breast nipple orifice and comprising:
an elongated overlay of a pliant material, defining a nipple aperture sized to receive the breast nipple, having a dorsal surface, a ventral surface, and an elongated window extending radially outwardly from the nipple aperture;
the overlay being contoured to provide a lingulate region adjacent to the nipple aperture, a mammary duct orifice map precursor situated in the lingulate region, and at least a portion of the dorsal surface of the overlay being a markable surface.

2. The device of claim 1 wherein the elongated window is a transparent region of the overlay.

3. The device of claim 1 wherein the elongated window is a slot in the overlay.

4. The device of claim 3 wherein the window is integrally formed with the nipple aperture.

5. The device of claim 1 wherein the overlay is provided with a slit that extends inwardly from a periphery of the overlay to the nipple aperture.

6. The device of claim 1 wherein the lingulate region is unitary with the overlay.

7. The device of claim 1 wherein the pliant material is transparent.

8. The device of claim 1 wherein the ventral surface of the overlay is provided with a pressure sensitive adhesive.

9. The device of claim 1 wherein distance indicia are provided on the overlay along the elongated window thereof.

10. The device of claim 1 wherein azimuth indicia are provided on the overlay about the nipple aperture.

11. The device of claim 1 wherein at least the ventral surface of the overlay is provided with an antimicrobial agent.

12. The device of claim 1, further including a patient portfolio.

13. The device of claim 12, wherein the patient portfolio comprises a plurality of photograph holders.

14. The device of claim 13, wherein a photograph of an introducer sheath angle in a relaxed breast is stored in at least one of the plurality of photograph holders.

15. The device of claim 12, wherein the patient portfolio comprises a pouch for storing the overlay.

16. The device of claim 12, wherein the patient portfolio includes a writable region for entering information about the patient.

17. A kit for mapping a mammary duct that terminates in a breast nipple orifice, the kit comprising:
an external target marker for marking an external surface of the breast;
a nipple alignment marker having at least one body part alignment indicator;
a pliant alignment slide overlay conformable to human breast contour and adapted for attachment to the nipple alignment marker; and
a mammary duct orifice map precursor adapted for attachment to the alignment slide overlay.

18. The kit of claim 17 wherein the external target marker includes an adhesive backing for removable attachment to the human breast.

19. The kit of claim 17 wherein the external target marker comprises a plurality of regions having different opacities.

20. The kit of claim 17 wherein the nipple alignment marker includes a first adhesive backing for removable attachment to the human breast proximate the nipple.

21. The kit of claim 20 wherein the nipple alignment marker includes a second adhesive backing for attachment to the alignment slide overlay.

22. The kit of claim 17 wherein the at least one body part alignment indicator is a marking for patient's head position.

23. The kit of claim 17 wherein the at least one body part alignment indicator has markings for patient's head position and patient's feet position.

24. The kit of claim 17 wherein the nipple alignment marker circumscribes the human nipple.

25. The kit of claim 17 wherein the nipple alignment marker is disc-shaped and defines a central opening sized to receive a breast nipple.

26. The kit of claim 17 wherein the mammary duct map precursor includes at least one body part alignment indicator.

27. The kit of claim 26 wherein the mammary duct map precursor further defines at least one window whereby the at least one body part alignment indicator of the nipple alignment marker can be aligned with the body part alignment indicator on the mammary duct map precursor.

28. The kit of claim 17 wherein the mammary duct map precursor is integral with the alignment slide.

29. The kit of claim 17 wherein the alignment slide and the mammary duct map precursor are adhesively secured to one another.

30. The kit of claim 17 wherein the nipple alignment marker includes a slit.

31. The kit of claim 17 wherein the alignment slide overlay includes a slit.

32. The kit of claim 17 wherein the mammary duct map precursor includes a slit.
